(19) United States
(12) Patent Application Publication
(10) Pub. No.: US 2006/0040962 A1
(43) Pub. Date: Feb. 23, 2006

(54) Pharmaceutical formulations

(75) Inventors: Zhiyun Wang, East Hanover, NJ (US); Surendra A. Sangekar, Union, NJ (US); Ping I. Lee, Toronto (CA)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 10/993,744

(22) Filed: Nov. 19, 2004

Related U.S. Application Data
(60) Provisional application No. 60/524,731, filed on Nov. 21, 2003.

(51) Int. Cl. A61K 31/522; A61K 9/14

(52) U.S. Cl. 514/263.35; 424/486

(57) ABSTRACT

Disclosed are pharmaceutically acceptable PDE V inhibitor Formulations that are especially useful for treating male erectile and female sexual dysfunction and other physiological disorders.

### CROSS-REFERENCE TO RELATED APPLICATION

This application claims benefit of priority to U.S. Provisional Patent Application Ser. No. 60/524,731, filed Nov. 21, 2003 the entirety of which is hereby incorporated by reference as if set forth fully herein.

### BACKGROUND OF THE INVENTION

The invention relates to polycyclic xanthine phosphodiesterase V inhibitors. Phosphodiesterase (“PDE”) V inhibitor compounds are described by Kenneth J. Murray in Phosphodiesterase V_{A} Inhibitors, DN & P 6(3), pp.150-156 (April, 1993), which is hereby incorporated herein by reference in its entirety, to have potential therapeutic value for a number of physiological disorders. One compound disclosed in the Murray article is MIMAX, a polycyclic xanthine PDE V inhibitor substituted at its 8-position with a —NHCH₃ group.

U.S. Pat. No. 5,409,934, which is hereby incorporated herein by reference in its entirety, discloses a series of xanthine PDE V inhibitors that are substituted at the 8-position with, among other possibilities, one of the following groups:—NO₂, —NR^{s}R^{t} or —NR⁶SO₂R⁵, where R^{s} and R^{t}, independently of one another, are each a hydrogen atom or an alkyl group, or R^{s} and R^{t}, together with the nitrogen atom to which they are both attached, form a phthalimido group, R⁵ is an alkyl or aryl group, and R⁶ is a hydrogen atom or —SO₂R⁷, where R⁷ is an alkyl or aryl group.

U.S. Pat. No. 5,470,579, which is hereby incorporated herein by reference in its entirety, discloses a xanthine PDE V inhibitor having a substituted or unsubstituted —NH₂ group at the 8-position, for example, —NHR, where R is a C₁-C₆ alkyl group.

WO93/23401, which is hereby incorporated herein by reference in its entirety, discloses xanthine PDE V inhibitors that are substituted at the 8-position with —NH(CH₂)₂CH(CH₂OR⁴)₂.

WO92/05176, which is hereby incorporated herein by reference in its entirety, discloses 8-acylaminoxanthine PDE V inhibitors that are substituted at the 8-position with —NHCOC₆H₅COOH.

WO92/05175, which is hereby incorporated herein by reference in its entirety, discloses 8-aminoxanthine PDE V inhibitors that are substituted at the 8-position with —NH₂ or —NHR, where R is an alkyl, arylalkyl or unsaturated heterocyclic (e.g., heteroaryl) group.

Specific PDE V inhibitors have been found useful for specific indications. For example, the use of PDE V inhibitors for treating impotence has met with commercial success with the introduction of sildenafil citrate, better known as Viagra® (Pfizer, NY, N.Y.). The chemistry and use of Viagra®, including its mechanism of action in treating erectile dysfunction, are taught in EP 0 702 555 B1, which is hereby incorporated herein by reference in its entirety. Additional PDE V inhibitors useful for treating erectile dysfunction are disclosed in WO99/24433, which is hereby incorporated herein by reference in its entirety.

Erectile dysfunction is a treatable and highly recognized health concern, affecting more than 30 million men in the United States, including one in four over age 65. Erectile dysfunction occurs when a man consistently is unable to sustain an erection sufficient for conducting sexual intercourse. In the past, psychological reasons were the most common explanation for erectile dysfunction or it was considered a natural part of aging. However, researchers today acknowledge that more than 70 percent of instances of erectile dysfunction are due to physical or medical problems. There are several factors that may contribute to erectile dysfunction, including:
Poor blood circulation—atherosclerosis or hardening of the arteries, high blood pressure and high cholesterol.
Neurological disorders—multiple sclerosis, Alzheimer's disease and Parkinson's disease.
Hormone imbalances—diabetes, thyroid disorders and low testosterone levels.
Trauma—spinal cord injury, prostate surgery or other trauma to the pelvic area.
Prescription and over-the-counter medications—blood pressure medications, antidepressants and certain drug combinations.
Lifestyle habits—smoking, alcohol abuse and using illegal drugs.

U.S. Pat. Nos. 5,939,419 and 5,393,755, both of which are hereby incorporated herein by reference in their entirety, disclose polycyclic guanine PDE V derivatives that are useful for the treatment of cardiovascular and pulmonary disorders.

As has been shown by the representative art cited above, certain xanthine/guanine PDE V inhibitors have been found to be useful for treating cardiovascular and pulmonary disorders, while some others have been found useful for treating impotence. It has been further shown that certain xanthine PDE V inhibitors can be substituted at the 8-position by a variety of groups, including nitro and unsubstituted or substituted amino groups. The substituted amino groups include saturated heterocycles, where the nitrogen atom and its substituents together form an unsaturated heterocyclic group (e.g., —NR^{x}R^{y} can form a heterocycle).

U.S. patent application Ser. Nos. 09/940,760 and 60/315,395, filed Aug. 28, 2001, 60/344,498 filed Nov. 9, 2001, and 60/384,478 and 60/384,484 filed May 31, 2002, all of which are hereby incorporated by reference in their entirety, disclose novel compounds for the inhibition of PDE V enzymes as well as processes for producing such novel compounds.

One particular species disclosed therein, 7-[(3-Bromo-4-methoxyphenyl)methyl]-1-ethyl-3,7-dihydro-8-[[(1R,2R)-2-hydroxycyclopentyl]amino]-3-(2-hydroxyethyl)-1H-purine-2,6-dione, has been found to be particularly effective as a PDE V inhibitor. However, the compound has poor solubility in water and poor wettability in its crystalline form. As a result, the absorption of the compound from the gastrointestinal tract is slow due to its slow dissolution rate. Additionally, tests conducted so far in animals indicate that the bioavailability of the compound is also low.

Accordingly, there exists a need for Formulations of the above described PDE V inhibitor compounds that provide enhanced bioavailability of the compounds. There also exists a need for a Formulation of the above PDE V inhibitor compounds that can be manufactured in a tablet or capsule form that has improved bioavailability. Thus, this invention overcomes the problem of making active compounds that have a very low aqueous solubility to be more bioavailable.

### SUMMARY OF THE INVENTION

Accordingly, it is an object of the present invention to provide a pharmaceutically acceptable composition comprising 7-[(3-Bromo-4-methoxyphenyl)methyl]-1-ethyl-3,7-dihydro-8-[[(1R,2R)-2-hydroxycyclopentyl]amino]-3-(2-hydroxyethyl)-1H-purine-2,6-dione, including an enantiomer, stereoisomer, rotomer, tautomer and/or prodrug thereof, in combination with a polymeric carrier and a wetting agent to form a tri-component co-precipitate composition.

It is also an object of the present invention to provide a pharmaceutical composition comprising a substantially amorphous high energy dispersion, said high energy dispersion comprising: a pharmaceutically active ingredient comprising a compound having the Formula: 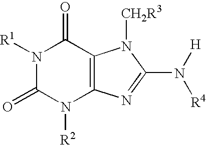
where,
(a) R¹ and R² are, independently of one another, each a C₁₋₁₅ alkyl group, branched or straight chain, with or without one or more substituents, a C₂₋₁₅ alkenyl group, branched or straight chain, with or without one or more substituents, a C₂₋₁₅ alkynyl group, branched or straight chain, with or without one or more substituents, a C₃₋₁₅ cycloalkyl group, with or without one or more substituents, an arylalkyl group, with or without one or more substituents, an aryl group, with or without one or more substituents, a heteroaryl group, with or without one or more substituents, —OR⁵, —COOR⁵, —C(O)R⁵ or —C(O)N(R⁵)₂, where, R⁵ is a hydrogen atom or a hydrocarbon radical, with or without one or more substituents, or one of R¹ and R² is a hydrogen atom and the other one of R¹ and R² is defined the same as above;
(b) R³ is an aryl group, with or without one or more substituents, a heteroaryl group, with or without one or more substituents, or a heterocyclic group having 1 to 3 heteroatoms fused to a 5- or 6-membered aryl ring, with or without one or more substituents, with the proviso that R³ is not an aryl group substituted at its para position with a —Y-aryl group, where, Y is a carbon-carbon single bond, —CO—, —O—, —S—, —N(R²¹)—, —CON(R²²)—, —N(R²²)CO—, —OCH₂—, —CH₂O—, —SCH₂—, —CH₂S—, —NHC(R²³)(R²⁴)—, —NR²³SO₂—, —SO₂NR²³—, —(R²³)(R²⁴)NH—, —CH═CH—, —CF═CF—, —CH═CF—, —CF═CH—, —CH₂CH₂—, —CF₂CF₂—, 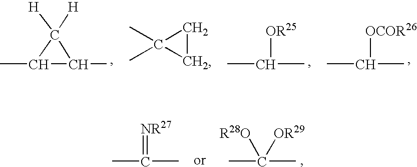
   where,
R²¹ is a hydrogen atom or a —CO(C₁₋₄ alkyl), C₁₋₆ alkyl, allyl, C₃₋₆ cycloalkyl, phenyl or benzyl group;
R²² is a hydrogen atom or a C₁₋₆ alkyl group;
R²³ is a hydrogen atom or a C₁₋₅ alkyl, aryl or —CH₂-aryl group;
R²⁴ is a hydrogen atom or a C₁₋₄ alkyl group;
R²⁵ is a hydrogen atom or a C₁₋₈ alkyl, C₁₋₈ perfluoroalkyl, C₃₋₆ cycloalkyl, phenyl or benzyl group;
R²⁶ is a hydrogen atom or a C₁₋₆ alkyl, C₃₋₆ cycloalkyl, phenyl or benzyl group;
R²⁷is —NR²³R²⁴, —OR²⁴, —NHCONH₂, —NHCSNH₂, 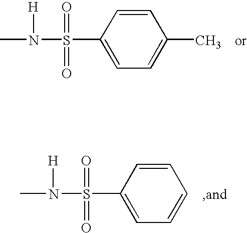
R²⁸ and R²⁹ are, independently of one another, each a C₁₋₄ alkyl group or, taken together with each other, a —(CH₂)_{q} group, where q is 2 or 3; and
(c) R⁴ is a C₃₋₁₅ cycloalkyl group, with or without one or more substituents, a C₃₋₁₅ cycloalkenyl group, with or without one or more substituents, or a heterocycloalkyl group of 3 to 15 members, with or without one or more substituents;

wherein, the one or more substituents for all the groups are chemically-compatible and are, independently of one another, each an: alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo, nitro, oximino, —COOR⁵⁰, —COR⁵⁰, —SO₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO(R⁵⁰), —N(R⁵²)COOR⁵⁰ or —N(R⁵²)CON(R⁵⁰R⁵¹) group, where:
R⁵⁰, R⁵¹ and R⁵² are, independently of one another, each a hydrogen atom or a branched or straight-chain, optionally substituted, C₁₋₆ alkyl, C₃₋₆ cycloalkyl, C₄₋₆ heterocycloalkyl, heteroaryl or aryl group, or R⁵⁰ and R⁵¹ are joined together to form a carbocyclic or heterocyclic ring system, or R⁵⁰, R⁵¹ and R⁵² are, independently of one another, each: 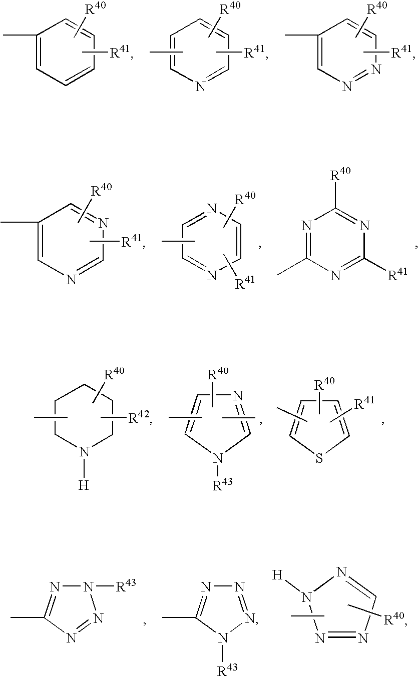
   where,
R⁴⁰ and R⁴¹ are, independently of one another, each a hydrogen atom or a branched or straight-chain, optionally substituted, alkyl, cycloalkyl, heterocycloalkyl, halo, aryl, imidazolyalkyl, indolyalkyl, heteroaryl, arylalkyl, arylalkoxy, heteroarylalkyl, heteroarylalkoxy, aminoalkyl, haloalkyl, mono-, di- or trihaloalkyl, mono-, di- or trihaloalkoxy, nitro, cyano, alkoxy, hydroxy, amino, phosphino, phosphate, alkylamino, dialkylamino, formyl, alkylthio, trialkylsilyl, alkylsulfonyl, arylsulfonyl, alkylsulfinyl, aminoalkyl, alkylaminoalkyl, dialkylaminoalkyl, hydroxyalkyl, morpholino, thioalkyl, alkylthioalkyl, carboxyalkyl, oximino, —COOR⁵⁰, —COR⁵⁰, —SO₀₋₂R⁵⁰, —SO₂NR⁵⁰R⁵¹, —NR⁵²SO₂R⁵⁰, —CON(R⁵⁰R⁵¹), —OCON(R⁵⁰R⁵¹), —N(R⁵²)CO(R⁵⁰), —N(R⁵²)COOR⁵⁰, —N(R⁵²)CON(R⁵⁰R⁵¹) or —OCONR⁵⁰ group, where, R⁵⁰, R⁵¹ and R⁵² are defined the same as above;
R⁴² is a hydrogen atom or a branched or straight-chain, optionally substituted, alkyl, alkenyl, arylalkyl or acyl group; and
R⁴³ is a hydrogen atom or a branched or straight-chain, optionally substituted, alkyl or aryl group;

wherein, the optional substituents are defined the same as above for the one or more substituents in admixture with a polymer matrix comprising a polymeric carrier and a wetting agent to form a high energy dispersion, wherein the ratio of the pharmaceutically active ingredient to the polymer matrix is about 1:1 to about 1:10.

It is also an object of the present invention to provide a method for treating a patient suffering from a PDE V disorder, such as erectile dysfunction, comprising administering a medication comprising 7-[(3-Bromo-4-methoxyphenyl)methyl]-1-ethyl-3,7-dihydro-8-[[(1R,2R)-2-hydroxycyclopentyl]amino]-3-(2-hydroxyethyl)-1H-purine-2,6-dione, including an enantiomer, stereoisomer, rotomer, tautomer and/or prodrug thereof, to said patient to diminish the symptoms of said disorder.

These and other objects of the invention will become apparent as the description progresses.

### BRIEF DESCRIPTION OF THE FIGURES

FIG. 1 is the mean plasma concentration of the active ingredient suspended in 0.4% HPMC over time at a dose of 50 mg.

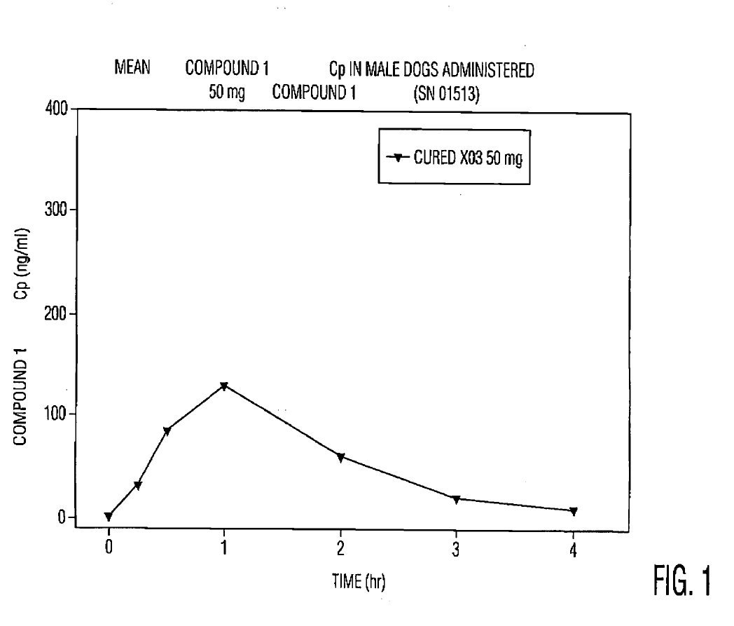

FIG. 2 is the mean plasma concentration of the active ingredient over time at a dose of 50 mg for high energy dispersion formulations.

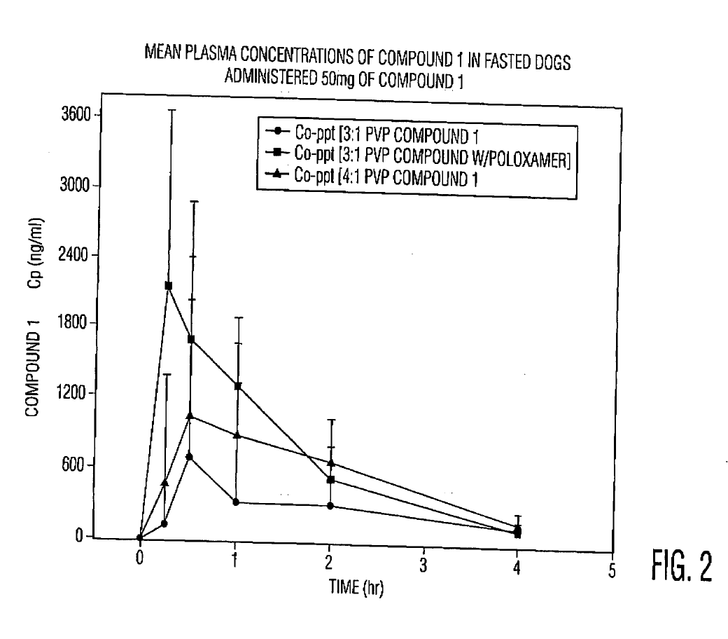

### WRITTEN DESCRIPTION OF THE INVENTION

Unless stated otherwise, wt % is based on the total weight of the composition such that the sum equals 100 wt %.

7-[(3-Bromo-4-methoxyphenyl)methyl]-1-ethyl-3,7-dihydro-8-[[(1R,2R)-2-hydroxycyclopentyl]amino]-3-(2-hydroxyethyl)-1H-purine-2,6-dione is a phosphodiesterase inhibitor with specificity for the PDE V isozyme. Phosphodiesterase inhibition potentiates the action of cyclic guanosine monophosphate (cGMP) by sparing cGMP the catabolic action of the enzyme. cGMP causes smooth muscle relaxation and influx of blood to the corpus cavernosum, facilitating erection. This mechanism of action makes this compound a useful compound in the treatment of erectile dysfunction.

7-[(3-Bromo-4-methoxyphenyl)methyl]-1-ethyl-3,7-dihydro-8-[[(1R,2R)-2-hydroxycyclopentyl]amino]-3-(2-hydroxyethyl)-1H-purine-2,6-dione has the following chemical structure as set forth in Formula I:

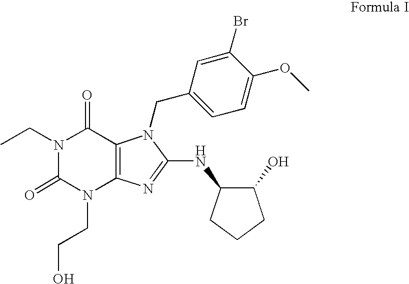

The compound of Formula I can exist in various polymorphic forms. For instance, Form I of the above compound is a needle shaped crystalline material. Form II, for instance, is a plate shaped crystalline form. The above compound may also exist in an amorphous state. Finally, the above compound may exist as a mixture of crystalline and amorphous material.

Preferably, the above compound is present in the pharmaceutical composition in an amount of about 1 mg to about 200 mg, or about 1 mg to about 100 mg, or about preferably about 5 to about 100 mg.

Formulations of the present invention combine 7-[(3-Bromo-4-methoxyphenyl)methyl]-1-ethyl-3,7-dihydro-8-[[(1R,2R)-2-hydroxycyclopentyl]amino]-3-(2-hydroxyethyl)-1H-purine-2,6-dione with a polymer system composed of polymers selected from the group consisting of: povidone, such as povidone K30, povidone K12, povidone K90, crospovidone, hydroxypropyl methylcellulose, hydroxypropylcellulose, polyethylene oxide, gelatin, carbomer, carboxymethylcellulose, methylcellulose, cellulose acetate phthalate, hydroxypropylmethylcellulose phthalate and propylene glycol alginate; and a wetting agent selected from the group comprising polysorbate 80, polaxamer 188, polaxamer 124, wherein the ratio of said compound to said polymer system is about 1:1 to about 1: 10, preferably about 1:3 to about 1:6.

The term “high energy dispersion” describes a homogeneous solution of the PDE V inhibitor of Formula I in a polymer matrix, including soluble polymers (e.g., the compound of Formula I with povidone) and/or insoluble polymers (e.g., the compound of Formula I with crospovidone, wherein the PDE V inhibitor is molecularly dispersed in the polymer matrix). For instance, the high energy dispersion of the PDE V inhibitor of Formula I is prepared by dissolving the PDE V inhibitor and a soluble polymer in a suitable organic solvent and then removing the solvent to give a high energy dispersion. The high energy dispersion is a homogeneous amorphous matrix of the PDE V inhibitor and the polymer. For example, a high energy dispersion of the PDE V inhibitor, povidone and polysorbate 80. High energy dispersion, molecular dispersion and co-precipitate mean the same thing and may be used interchangeably as is known to one of skill in the art.

Alternatively, the high energy dispersion can be produced by dissolving the PDE V inhibitor of Formula I in a suitable organic solvent that will swell an insoluble polymeric matrix, and then absorbing the resulting solution into the insoluble polymeric matrix. The solvent is then evaporated from the resulting mixture. This results in a high energy dispersion that is essentially in an amorphous state wherein the PDE V inhibitor is molecularly dispersed in the polymeric matrix, such as crospovidone.

Additional methods of preparation of high energy dispersion include dissolving the PDE-V inhibitor, polymeric materials and additives in organic solvent and pouring solution onto a substrate to cast film. Alternatively, the solution can be sprayed onto perial beads or surfaces of tablets. After evaporation of organic solvent, the thin film is formed that is comprised of the high energy dispersion. The solution alternatively can be spray dried using a suitable spray dryer to give powder.

A non-solvent system can also be used for preparation of the high energy dispersion through hot melt extrusion. The PDEV inhibitor plus polymeric materials and additives are mixed and fed into extruder that is programmed at appropriate temperature, pressure and speed. This process causes melting of the crystalline form of PDEV inhibitor to form amorphous drug substance that is stabilized by the presence of polymeric materials.

Alternatively, high energy dispersion can also be prepared by application of super critical fluid that forms amorphous drug substance in the presence of polymeric matrix. The drug substance, polymer and surfactant or/and other additives are dissolved in suitable solvent or solvents. The solution is then injected into the super critical fluid, i.e., carbon dioxide. The precipitated high energy dispersion will be collected.

Suitable polymers for use as the polymeric matrix in the high energy dispersion are selected from the group consisting of povidone, crospovidone, hydroxypropyl methylcellulose, hydroxypropyl-cellulose, polyethylene oxide, gelatin, carbomer, carboxymethyl-cellulose, croscarmellose, methylcellulose, ammonio methacrylate copolymer, cellulose acetate phthalate, hydroxypropylmethylcellulose phthalate and propylene glycol alginate. Crospovidone and croscarmellose are insoluble polymers; povidone, hydroxypropylmethylcellulose, hydroxypropylcellulose, polyethylene oxide, gelatin, carbomer, carboxymethylcellulose, methylcellulose, ammonio methacrylate copolymer, cellulose acetate phthalate, hydroxypropylmethylcellulose phthalate and propylene glycol alginate are soluble polymers. Preferably, povidone or crospovidone is used; and more preferably, povidone is used.

Povidone represents 1-vinyl-2-pyrrolidinone polymers (polyvinylpyrrolidone) having a molecular weight average ranging from about 2,500 to about 1,000,000, preferably in a range of from about 3,000 to about 74,000,

Crospovidone represents water-insoluble synthetic cross-linked homopolymers of N-vinyl-2-pyrrolidinone. Generally, the crospovidone has a particle size of about 20 μM to about 250 μM, and preferably about 50 μM to about 250 μM (see, for example, Kollidon, polyvinylpyrrolidone for the pharmaceutical industry by BASF).

Suitable solvents for the polymer matrix include methanol, ethanol, acetone, isopropyl alcohol or a combination of the above solvents.

The ratio of the 7-[(3-Bromo-4-methoxyphenyl)methyl]-1-ethyl-3,7-dihydro-8-[[(1R,2R)-2-hydroxycyclopentyl]amino]-3-(2-hydroxyethyl)-1H-purine-2,6-dione to polymer matrix is about 1:1 to about 1:10, preferably about 1:4 to about 1:6 and more preferably about 1:3.

In the Formulations of the present inventions, it is preferred to maintain the 7-[(3-Bromo-4-methoxyphenyl)methyl]-1-ethyl-3,7-dihydro-8-[[(1R,2R)-2-hydroxycyclopentyl]amino]-3-(2-hydroxyethyl)-1H-purine-2,6-dione in its amorphous state. The conversion of the amorphous drug to its crystalline state is greatly retarded due to the presence of large quantity of povidone K30, or similar polymers, that have a high glass transition temperature. The presence of small quantity of surfactant improves wetting of the drug and reduces static buildup in the resulting product for proper handling. Key parameters affecting the properties of present high-energy dispersion and to the properties of final product include: ratio of drug to povidone K30, polysorbate 80 concentration, solvent type, conditions in preparation of solution and method of solvent evaporation. By amorphous, it is meant that there was no detection of crystalline drug by x-ray diffraction or differential scanning calorimetry.

The dosage form comprising the high energy dispersion can, optionally, further comprise additional excipients suitable for use in either tablet or capsule form selected from the group comprising: diluents, disintegrants, lubricants, surfactants, glidants, artificial sweeteners, bulking agents, colorants and one or more flavorants. Generally, the composition comprising the high energy dispersion into tablet and capsule dosage forms can, optionally, further comprise: about 8 to about 40 wt % of one or more disintegrants, about 0.5 to about 2 wt % of one or more lubricants, about 4 to about 10 wt % of one or more surfactants, about 0.5 to about 5 wt % of one or more glidants; about 1 to about 10 wt % of one or more artificial sweeteners, about 40 to about 60 wt % of one or more bulking agents, about 0.1 to about 10 wt % of one or more colorants (coloring agents), and/or about 1 to about 5 wt % of one or more flavorants (flavoring agents).

This invention also provides solid dosage forms comprising the high energy dispersion described above. Solid dosage forms include tablets, capsules and chewable tablets. Excipients that are pharmaceutically generally considered safe can be blended with the solid solution to provide the desired dosage form. For example, a capsule can contain the solid solution blended with (a) a disintegrant and a lubricant, or (b) a disintegrant, a lubricant and an additional surfactant. A tablet can contain the solid solution blended with at least one disintegrant, a lubricant, a surfactant, and a glidant. The fast dissolving or buccal tablet can contain the solid solution blended with a bulking agent, a lubricant, and if desired an additional sweetening agent (such as an artifical sweetener), and suitable flavors.

Suitable disintegrants are selected from the group comprising: croscarmellose sodium (a cross linked polymer of carboxymethylcellulose sodium, see NF XVII page 1922 (1990)), crospovidone, starches, celluloses, alginates, and gums. Preferably, the disintegrant is selected from croscarmellose sodium or crospovidone. Preferably, croscarmellose sodium is used as the disintegrant in compositions for capsules. Preferably, crospovidone is used as the disintegrant in compressible tablets. Those skilled in the art will appreciate that it is desirable for compressible tablets to disintegrate within 5-15 minutes; therefore, the disintegrant used preferably results in the disintegration of the tablet within 5-15 minutes.

Suitable lubricants include talc, magnesium stearate, calcium stearate, stearic acid, hydrogenated vegetable oils and the like. Preferably, magnesium stearate is used.

Suitable surfactants include polyether glycols such as Pluronic® F-68 (Poloxamer 188 a block copolymer of ethylene glycol and propylene glycols), Pluronic® F87 (Poloxamer 237), Pluronic® F108 (Poloxamer 338), Pluronic® F127 (Poloxamer 407) and the like. Preferably, Pluronic® F-68 is used. According to BASF Corporation's Technical Bulletin (1995), Pluronic® is a registered tradename for BASF Corporation's block copolymers of ethylene oxide and propylene oxide represented by the chemical structure HO(C₂H₄O)ₐ(C₃H₆O)_{b}(C₂H₄O)ₐH wherein for: (a) Pluronic® F-68, a is 80 and b is 27; (b) Pluronic® F87, a is 64 and b is 37; (c) Pluronic® F108, a is 141 and b is 44; and Pluronic® F127, a is 101 and b is 56. The average molecular weights for these block copolymers are: (a) Pluronic® F-68, 8400; (b) Pluronic® F87, 7700; (c) Pluronic® F108, 14600; and Pluronic® F127, 12600.

Suitable bulking agents include xylitol, mannitol, compressible sugars, lactose, and microcrystalline celluloses.

Suitable artificial sweeteners include saccharin, cyclamates and aspartame.

If desired known flavorants and known FD & C colorants can be added to the composition.

For capsule dosage forms, the composition comprising the high energy dispersion generally further comprises diluents, disintegrants, lubricants, and, optionally, surfactants. Thus, a composition for use in capsules can comprise about 10 to about 90 wt % of the high energy dispersion, about 8 to about 20 wt % of one or more disintegrants, about 0.5 to about 2 wt % of one or more lubricants, and, optionally, about 4 to about 10 wt % of one or more surfactants, about 10 to about 90 % diluent or a combination of diluents.

For example, a composition for use in a capsule dosage form comprises: about 80 to about 90 wt % of the high energy dispersion, about 8 to about 20 wt % of one or more disintegrants and about 0.5 to about 2 wt % of one or more lubricants, and about 10 to about 90 % diluent or diluents.

Another example of a composition for use in a capsule dosage form is a composition comprising about 80 to about 90 wt % of the high energy dispersion, about 8 to about 15 wt % of one or more disintegrants, about 0.5 to about 2 wt % of one or more lubricants, and about 4 to about 10 wt % of one or more surfactants, and about 10 to about 90 % diluent or diluents.

In general, the compositions for capsule dosage forms contain the high energy dispersion, one diluent, one disintegrant, one lubricant, and optionally, one surfactant. The surfactant, in particular Pluronic F-68, is an important ingredient that significantly enhanced the oral bioavailability of the capsule product and reduced subject to subject variability based on animal studies.

For a compressible tablet dosage form the composition comprising the high energy dispersion generally further comprises diluents, disintegrants, lubricants, surfactants, and glidants. Thus, a composition for use in compressible tablets can comprise about 30 to about 70 wt % of the high energy dispersion, about 20 to about 60% diluent, about 5 to about 40 wt % of one or more disintegrants, about 0.5 to about 2 wt % of one or more lubricants, about 2 to about 10 wt % of one or more surfactants, and about 1 to about 2 wt % of one or more glidants. Preferably, the disintegrant is croscarmellose sodium.

In addition to the disintegrant, the compressible tablet also preferably comprises one diluent, one lubricant, one surfactant and one glidant.

For chewable tablets, the composition generally comprises about 40 to about 60 wt % of the high energy dispersion, about 40 to about 60 wt % of a bulking agent (e.g., a sugar such as xylitol, mannitol), and about 0.5 to about 2 wt % of a lubricant, optionally about 1 to about 10 wt % of an artificial sweetener (e.g., sodium saccharin or aspartame), and optionally about 0.1 to about 10 wt % of a colorant.

Other preferred diluents include lactose, mannitol, sorbitol, tribasic calcium phosphate, diabasic calcium phosphate, compressible sugar, starch, calcium sulfate, dextro and microcrystalline cellulose. Pharmaceutical compositions of the invention generally contain from about 0 to 75% of diluents.

Preferred lubricants/glidants may include magnesium stearate, stearic acid and talc. Pharmaceutical compositions of the invention generally include from about 0.5 to 7% , preferably, about 0.5 to 5% of lubricants/glidants.

Preferred disintegrants may include starch, sodium starch glycolate, crospovidone and croscarmelose sodium and microcrystalline cellulose.

Pharmaceutical compositions of the invention generally include from about 0 to 20% , preferably, about 4 to 15% of disintegrants.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of Formula I or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective that produces the desired therapeutic, ameliorative or preventative effect.

Compounds of the present invention, and salts and solvates thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts and solvates of the compounds), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate,” “prodrug” and the like, is intended to equally apply to the salt, solvate and prodrug of enantiomers, stereoisomers, rotamers, tautomers, racemates or prodrugs of the inventive compounds.

“Co-crystal” means a crystalline structure simultaneously comprising pharmaceutically active molecules and inert molecules. Co-crystals may be formed by combining a weak base with a weak acid selected to match hydrogen bond donors with acceptors. The pKa difference of conjugate pairs may be inconsistent with salt formation in water. The co-crystallizing agents used to form co-crystals are usually bifunctional acids such as fumaric acid, succinic acid, malic acid, and tartaric acid. Co-crystals are discussed in J. F. Remenar et. al., “Crystal Engineering of Novel Cocrystals of a Triazole Drug with 1,4-Dicarboxylic Acids”, Journal of the American Chemical Society, 2003, vol. 125, pp. 8456-8457.

Another aspect of this invention is a method of treating a patient (e.g., human) having a disease or condition by administering a therapeutically effective amount of the compound of Formula I or a pharmaceutically acceptable salt or solvate, of said compound to the patient.

The invention will be more specifically set forth with the following non-limiting examples.

### EXAMPLE 1

The following compositions were prepared. The Povidone K30, polysorbate 80 and active were dissolved in methanol that was pre-heated to a temperature of 50 to 90 degrees Celsius. The resulting methanol solution was then sprayed into a stream of hot nitrogen so as to allow rapid evaporation of the methanol solvent. This process produced a fine powder in which the amorphous active ingredient (compound of Formula 1) is embedded in the resulting povidone K30 and polysorbate 80 matrix. The spray-dried high-energy dispersion was further dried to reduce the residual organic solvent level-to below 0.1% (g/g). Next, the preparation was blended with microcrystalline cellulose, poloxamer 188, croscarmellose sodium and magnesium stearate in a high intensity mixing device and formed a homogeneous powder blend. The powder blend was then filled into hard gelatin capsules.

| High Energy Dispersion | Amounts | | |
|---|---|---|---|
| high energy dispersion | 200.0 | 200.0 | — |
| drug to polymer ratio = 1:3 | | | |
| high energy dispersion | — | — | 250.0 |
| drug to polymer ratio = 1:4 | | | |
| (1:4) | | | |
| Microcrystalline Cellulose | 92.8 | 76.8 | 104.0 |
| (Avicel PH 102) | | | |
| Silicon Dioxide | 3.2 | 3.2 | 4.0 |
| Croscarmellose Sodium | 22.4 | 22.4 | 40.0 |
| Poloxamer 188 | — | 16.0 | — |
| Magnesium stearate | 1.6 | 1.6 | 2.0 |
| TOTAL | 320.0 | 320.0 | 400.0 |

The following study has been conducted to determine the pharmacokinetics of three prototype Formulations of PDE V inhibitor active agent in co-precipitate capsules following a single oral dose of 50 mg of active to male beagle dogs.

| | | | Target | Test Article | |
|---|---|---|---|---|---|
| | | Dose | Dose | Concentra- | |
| Group | Test Article | Route^{a} | (mg/kg)^{b} | tion (mg) | Fasted |
| 1 | Active (Co-ppt | Oral | 5 | 50 | yes |
| | 1:3 capsule) | | | | |
| 2 | Active (Co-ppt | Oral | 5 | 50 | yes |
| | 1:3 + Poloxamer | | | | |
| | capsule) | | | | |
| 3 | Active (Co-ppt | Oral | 5 | 50 | yes |
| | 1:4 capsule) | | | | |
| Dogs were fasted overnight before dosing and for 4 hr after dosing. Water was available continuously. Dogs were dosed with a single tablet of PDE inhibitor active agent. Blood samples (˜2 mL) was collected into Vacutainer ® tubes containing EDTA from the jugular veins at the following | | | | | |
| # timepoints: 0 (pre-dose), 0.25, 0.5, 1, 2 and 4 hr post-dose. The samples were centrifuged for 10 minutes at approximately 2000 g in a refrigerated centrifuge maintained at approximately 4° C. The plasma was separated, transferred to plastic tubes and stored at −70° C. prior to analysis. | | | | | |

### EXAMPLE 2

Active of 11.7 kg and Povidone K30 of 11.1 kg and 0.15 kg of polysorbate 80 were dissolved in methanol at 50-90 degrees C. The solution was spray-dried using a suitable spray-drier equipment under nitrogen. The high energy dispersion was collected.

The high energy dispersion of 101 gram was blended with 2.5 g of silicon dioxide, 113 g of microcrystalline cellulose, 20 g of croscarmellose sodium, 12.5 g of poloxamer and 1.25 g of magnesium stearate. The homogenous blend was filled in size 1 capsule.

| Composition | mg/capsule |
|---|---|
| Active | 25 |
| Povidone K30 | 75 |
| polysorbate 80 | 1 |
| silicon dioxide | 2.5 |
| microcrystalline cellulose | 112.75 |
| croscarmellose sodium | 20 |
| poloxamer 188 | 12.5 |
| magnesium stearate | 1.25 |
| No. 1 hard gelatin capsule shell | 1 ea |

Although certain presently preferred embodiments of the invention have been described-herein, it will be apparent to those skilled in the art to which the invention pertains that variations and modifications of the described embodiments may be made without departing from the spirit and scope of the invention. Accordingly, it is intended that the invention be limited only to the extent required by the appended claims and the applicable rules of law.

What is claimed is:

1. A pharmaceutical composition comprising a substantially amorphous high energy dispersion, said high energy dispersion comprising: a pharmaceutically active ingredient represented by the structural Formula 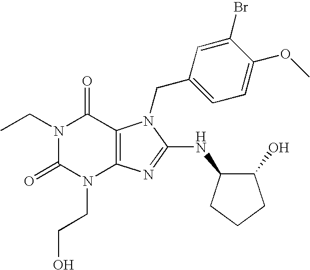
in admixture with a polymer matrix comprising a polymeric carrier and a wetting agent, wherein the ratio of the pharmaceutically active ingredient to the polymer matrix is about 1:1 to about 1:10.

2. The composition according to claim 1, wherein the polymeric carrier is povidone.

3. The composition according to claim 2, wherein the povidone has a molecular weight in a range of about 3000 to about 1,000,000.

4. The composition according to claim 3, wherein the povidone has a molecular weight in a range of about 3000 to about 9000.

5. The composition according to claim 1, wherein the povidone is povidone K30.

6. The composition according to claim 1, wherein the povidone is present in an amount of about 30% to about 90% .

7. The composition according to claim 1, wherein the wetting agent is selected from the group consisting of polysorbate 80 and Pluronic F-68.

8. The composition according to claim 7, wherein the polysorbate 80 is present in an amount of about 0.5% to about 3% .

9. The composition according to claim 7, wherein the Pluronic F-68 is present in an amount of about 3% to about 10% .

10. The composition according to claim 1, wherein the pharmaceutically active ingredient is present in an amount of about 1 to about 200 mg.

11. The composition according to claim 10, wherein the pharmaceutically active ingredient is present in an amount of about 5 mg.

12. The composition according to claim 10, wherein the pharmaceutically active ingredient is present in an amount of about 25 mg.

13. The composition according to claim 10, wherein the pharmaceutically active ingredient is present in an amount of about 50 mg.

14. The composition according to claim 10, wherein the pharmaceutically active ingredient is present in an amount of about 100 mg.

15. The composition according to claim 1, wherein the ratio of the pharmaceutically active ingredient to the polymer matrix is about 1:1 to about 1:6.

16. The composition according to claim 1, wherein the ratio of the pharmaceutically active ingredient to the polymer matrix is about 1:3.

17. The composition according to claim 1, further comprising a disintegrant, a lubricant and a diluent.

18. The composition according to claim 16, wherein the disintegrant is selected from the group consisting of croscarmelose sodium and crospovidone.

19. The composition according to claim 16, wherein the lubricant is selected from the group consisting of magnesium stearate and stearic acid.

20. The composition according to claim 16, wherein the diluent is selected from the group consisting of microcrystalline cellulose, lactose and mannitol.

21. A pharmaceutical composition comprising a substantially amorphous high energy dispersion, said high energy dispersion comprising: a pharmaceutically active ingredient comprising a compound having the Formula: 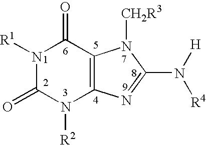
where,
(d) R¹ and R² are, independently of one another, each a C₁₋₁₅ alkyl group, branched or straight chain, with or without one or more substituents, a C₂₋₁₅ alkenyl group, branched or straight chain, with or without one or more substituents, a C₂₋₁₅ alkynyl group, branched or straight chain, with or without one or more substituents, a C₃₋₁₅ cycloalkyl group, with or without one or more substituents, an arylalkyl group, with or without one or more substituents, an aryl group, with or without one or more substituents, a heteroaryl group, with or without one or more substituents, —OR⁵, —COOR⁵, —C(O)R⁵ or —C(O)N(R⁵)₂, where, R⁵ is a hydrogen atom or a hydrocarbon radical, with or without one or more substituents, or one of R¹ and R² is a hydrogen atom and the other one of R¹ and R² is defined the same as above;
(e) R³ is an aryl group, with or without one or more substituents, a heteroaryl group, with or without one or more substituents, or a heterocyclic group having 1 to 3 heteroatoms fused to a 5- or 6-membered aryl ring, with or without one or more substituents, with the proviso that R³ is not an aryl group substituted at its para position with a —Y-aryl group, where, Y is a carbon-carbon single bond, —CO—, —O—, —S—, —N(R²¹)—, —CON(R²²)—, —N(R²²)CO—, —OCH₂—, —CH₂O—, —SCH₂—, —CH₂S—, —NHC(R²³)(R²⁴)—, —NR²³SO₂—, —SO₂NR²³—, (R²³)(R²⁴)NH—, —CH═CH—, —CF═CF—, —CH═CF—, —CF═CH—, —CH₂CH₂—, —CF₂CF₂—, 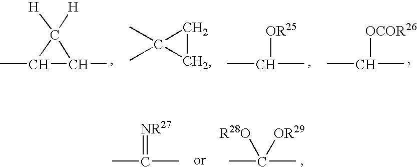
where,
R²¹ is a hydrogen atom or a —CO(C₁₋₄ alkyl), C₁₋₆ alkyl, allyl, C₃₋₆ cycloalkyl, phenyl or benzyl group;
R²² is a hydrogen atom or a C₁₋₆ alkyl group;
R²³ is a hydrogen atom or a C₁₋₅ alkyl, aryl or —CH₂-aryl group;
R²⁴ is a hydrogen atom or a C₁₋₄ alkyl group;
R²⁵ is a hydrogen atom or a C₁₋₈ alkyl, C₁₋₈ perfluoroalkyl, C₃₋₆ cycloalkyl, phenyl or benzyl group;
R²⁶ is a hydrogen atom or a C₁₋₆ alkyl, C₃₋₆ cycloalkyl, phenyl or benzyl group;
R²⁷ is —NR²³R²⁴, —OR²⁴, —NHCONH₂, —NHCSNH₂, 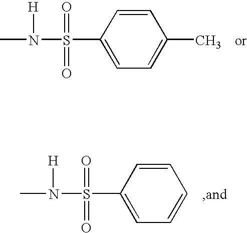
R²⁸ and R²⁹ are, independently of one another, each a C₁₋₄ alkyl group or, taken together with each other, a —(CH₂)_{q} group, where q is 2 or 3; and
(f) R⁴ is a C₃₋₁₅ cycloalkyl group, with or without one or more substituents, a C₃₋₁₅ cycloalkenyl group, with or without one or more substituents, or a heterocycloalkyl group of 3 to 15 members, with or without one or more substituents;
wherein, the one or more substituents for all the groups are chemically-compatible compatabile and are, independently of one another, each an: alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo, nitro, oximino, —COOR⁵⁰, —COR⁵⁰, —SO₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO(R⁵⁰), —N(R⁵²)COOR⁵⁰ or —N(R⁵²)CON(R⁵⁰R⁵¹) group, where:
R⁵⁰, R⁵¹ and R⁵² are, independently of one another, each a hydrogen atom or a branched or straight-chain, optionally substituted, C₁₋₆ alkyl, C₃₋₆ cycloalkyl, C₄₋₆ heterocycloalkyl, heteroaryl or aryl group, or R⁵⁰ and R⁵¹ are joined together to form a carbocyclic or heterocyclic ring system, or R⁵⁰, R⁵¹ and R⁵² are, independently of one another, each: 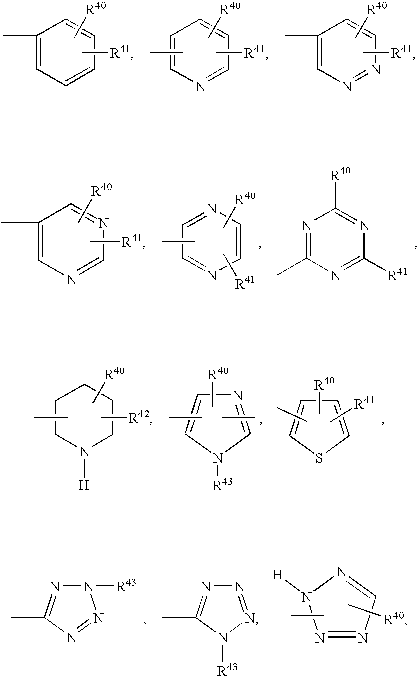
where,
R⁴⁰ and R⁴¹ are, independently of one another, each a hydrogen atom or a branched or straight-chain, optionally substituted, alkyl, cycloalkyl, heterocycloalkyl, halo, aryl, imidazolylalkyl, indolylalkyl, heteroaryl, arylalkyl, arylalkoxy, heteroarylalkyl, heteroarylalkoxy, aminoalkyl, haloalkyl, mono-, di- or trihaloalkyl, mono-, di- or trihaloalkoxy, nitro, cyano, alkoxy, hydroxy, amino, phosphino, phosphate, alkylamino, dialkylamino, formyl, alkylthio, trialkylsilyl, alkylsulfonyl, arylsulfonyl, alkylsulfinyl, aminoalkyl, alkylaminoalkyl, dialkylaminoalkyl, hydroxyalkyl, morpholino, thioalkyl, alkylthioalkyl, carboxyalkyl, oximino, —COOR⁵⁰, —COR⁵⁰, —SO₀₋₂R⁵⁰, —SO₂NR⁵⁰R⁵¹, —NR⁵²SO₂R⁵⁰, —CON(R⁵⁰R⁵¹), —OCON(R⁵⁰R⁵¹), —N(R⁵²)CO(R⁵⁰), —N(R⁵²)COOR⁵⁰, —N(R⁵²)CON(R⁵⁰R⁵¹) or —OCONR⁵⁰ group, where, R⁵⁰, R⁵¹ and R⁵² are defined the same as above;
R⁴² is a hydrogen atom or a branched or straight-chain, optionally substituted, alkyl, alkenyl, arylalkyl or acyl group; and
R⁴³ is a hydrogen atom or a branched or straight-chain, optionally substituted, alkyl or aryl group;
wherein, the optional substituents are defined the same as above for the one or more substituents;
in admixture with a polymer matrix comprising a polymeric carrier and a wetting agent, wherein the ratio of the pharmaceutically active ingredient to the polymer matrix is about 1:1 to about 1:10.

22. The composition according to claim 21, wherein the polymeric carrier is povidone.

23. The composition according to claim 22, wherein the povidone a molecular weight in a range of about 3000 to about 1,000,000.

24. The composition according to claim 23, wherein the povidone a molecular weight in a range of about 3000 to about 9000.

25. The composition according to claim 21, wherein the povidone is povidone K30.

26. The composition according to claim 21, wherein the povidone is present in an amount of about 30% to about 90% .

27. The composition-according to claim 21, wherein the wetting agent is selected from the group consisting of polysorbate 80 and Pluronic F-68.

28. The composition according to claim 27, wherein the polysorbate 80 is present in an amount of about 0.5% to about 3% .

29. The composition according to claim 27, wherein the Pluronic F-68 is present in an amount of about 3% to about 10% .

30. The composition according to claim 21, wherein the ratio of the pharmaceutically active ingredient to the polymer matrix is about 1:1 to about 1:6.

31. The composition according to claim 21, wherein the ratio of the pharmaceutically active ingredient to the polymer matrix is about 1:3.

32. The composition according to claim 21, further comprising a disintegrant, a lubricant and a diluent.

33. The composition according to claim 31, wherein the disintegrant is selected from the group consisting of croscarmelose sodium and crospovidone.

34. The composition according to claim 31, wherein the lubricant is selected from the group consisting of magnesium stearate and stearic acid.

35. The composition according to claim 31, wherein the diluent is selected from the group consisting of microcrystalline cellulose, lactose and mannitol.
